(19) United States
(12) Patent Application Publication
(10) Pub. No.: US 2017/0281694 A1
(43) Pub. Date: Oct. 5, 2017

(54) TOPICAL CLEANSING COMPOSITION WITH PREBIOTIC/PROBIOTIC ADDITIVE

(71) Applicant: GOJO Industries, Inc., Akron, OH (US)

(72) Inventors: Sarah Gantz, Akron, OH (US); Amanda Copeland, Seville, OH (US); Carrie Anne Zapka, Austintown, OH (US)

(21) Appl. No.: 15/475,705

(22) Filed: Mar. 31, 2017

Related U.S. Application Data
(60) Provisional application No. 62/316,316, filed on Mar. 31, 2016.

(51) Int. Cl. A61K 35/742; A61L 2/00; A61K 9/00

(52) U.S. Cl. CPC A61K 35/742; A61K 9/0014; A61L 2/0088; A61L 2202/20

(57) ABSTRACT

A topical cleansing composition is disclosed that includes an active ingredient, comprising one or more of a probiotic, a probiotic derivative, and prebiotic, a humectant, one or more surfactants; and water. Topical cleansing composition reduces pathogen binding on skin by a statistically significant amount, as compared to an otherwise identical topical composition without the active ingredient.

### RELATED APPLICATIONS

This application claims priority to and the benefit of U.S. Provisional Patent Application Ser. No. 62/316,316, entitled “TOPICAL CLEANSING COMPOSITION WITH PREBIOTIC/PROBIOTIC ADDITIVE” and filed Mar. 31, 2016, the entire disclosure of which is incorporated herein by reference.

### BACKGROUND

The skin is the human body's largest organ, colonized by a diverse range of microorganisms, the majority of which are harmless or even beneficial to their host. These microorganisms often provide vital functions that the human genome has not yet evolved to perform. In this way, the skin constantly regulates a balance between host-human and microorganism. Disruptions in this delicate balance, on either side, can result in serious skin disorders or infections.

Pathogens on the skin are known to cause illness and may be easily transmitted from one person to another. Some pathogens stick strongly to skin. Typically, when pathogens stick to skin, they are more difficult to remove or kill using traditional approaches to skin cleaning and disinfection such as washing with a conventional soap or waterless sanitizer. Pathogens that are stuck to skin are more dangerous because they remain on the skin longer. The longer the pathogen is on the skin, the more the chance that they will either cause infections on the person with them or be shared with other people.

The overuse of antibiotics is contributing an increase in the types and numbers of antibiotic-resistant pathogens, and infections from these pathogens are becoming more dangerous. There is an increasing interest in finding alternative ways to control pathogens without the use of more antimicrobials. Probiotics are being used to control microbes on skin in new ways that do not require the use of antimicrobials. Probiotics are live or inactivated microorganisms that, when either present as part of the normal microbiota or when administered in adequate amounts, confer a health or cosmetic benefit on the host. Benefits from probiotics can be from the microbial components directly or can come from the byproducts of bacterial growth.

Therefore, it would be beneficial to design a new soap and/or lotion composition that is safe for topical use and restores the natural balance of bacteria on the skin including decreasing the adherence of pathogens on the skin.

### SUMMARY

According to some exemplary embodiments, a topical cleansing composition for restoring skin's natural balance of bacteria is provided. The topical cleansing composition includes about 0.005 wt. % to 10.0 wt. % of an active ingredient, at least about 1.0 wt. % of one or more surfactants; and water. The active ingredient may comprise one or more of a probiotic, a probiotic derivative, and prebiotic. The topical cleansing composition reduces pathogen binding on skin by a statistically significant amount, as compared to an otherwise identical topical composition without the active ingredient.

In some exemplary embodiments, the active ingredient is a probiotic or probiotic derived ingredient, which can be selected from a strain of one or more the following families: Actinomycetaceae, Corynebacteriaceae, Nocardiaceae, Intrasporangiaceae, Micrococcaceae, Propionibacteriacea, Bacteroidaceae, Porphyromonadaceae, Flavobacteriaceae, Sphingobacteriaceae, Bacillaceae, Exiguobacteraceae, Gemellaceae, Planococcaceae, Staphlococcaceae, Carnobacteriaceae, Aeorcoccaceae, Lactobacillaceae, Acidaminacoccaceae, Clostridiaceae, Lachnospiraceae, Peptostreptococcaceae, Veillonellaceae, Caulobactereaceae, Acetobacteraceae, Rhodobacteriaceae, Bradyrhizobiaceae, Brucellaceae, Sphingomonadaceae, Comamonadaceae, Nei sseriaceae, Enterobaceriaceae, Pseudomonodaceae, Moraxellaceae, Pasteurellaceae, Xanthomonadaceae, Fusobacteriaceae, Chloroflexi, Chloroplasts, Cyanobacteria, and Streptophyta, for example. In some exemplary embodiments, the active ingredient is a probiotic or probiotic derived ingredient, which can be selected from a strain of one or more the following: Lactobacillus, strains and derivatives of Clostridia, strains and derivatives of Bifidobacterium, strains and derivatives of Saccharomyces, strains and derivatives of Lactococcus, strains and derivatives of Pedicoccus, strains and derivatives of Enterococcus, strains and derivatives of Escherichia, strains and derivatives of Alcaligenes, strains and derivatives of Corynebacterium, strains and derivatives of Bacillus, and strains and derivatives of Propionibacterium.

In some exemplary embodiments, the topical composition comprises 0.5 to 2 wt. %, or about 0.8 to about 1.5 wt. % active ingredient, based on the total weight of the topical composition. In some exemplary embodiments, the surfactant includes a mixture of primary and secondary surfactants.

In some exemplary embodiments, the topical composition comprises one or more humectants, selected from the group consisting of propylene glycol, hexylene glycol, 1,4-dihydroxyhexane, 1,2,6-hexanetriol, sorbitol, butylene glycol, caprylyl glycol, propanediol s, such as methyl propane diol, dipropylene glycol, triethylene glycol, glycerin (glycerol), polyethylene glycols, ethoxydiglycol, polyethylene sorbitol, and combinations thereof. The humectant may be present in an amount up to about 20 wt. %, based on the weight of the total composition.

Further exemplary embodiments relate to a method of skin treatment for stimulating the production of antimicrobial peptides on the skin. The method includes applying a topical cleansing composition to a skin surface, wherein the topical composition comprises about 0.005 wt. % to about 10.0 wt. % of an active ingredient, at least about 1.0 wt. % of one or more surfactants, about 0.01 wt. % to about 10.0 wt. % of a humectant, and water. The active ingredient comprises one or more of a probiotic, a probiotic derivative, and prebiotic. The topical composition reduces pathogen binding on skin by a statistically significant amount, as compared to an otherwise identical topical composition without the active ingredient. The method further includes rinsing the topical cleansing composition off with water.

Further exemplary embodiments relate to a topical cleansing composition for reducing skin irritation. The topical cleansing composition includes about 0.005 wt. % to about 10.0 wt. % of an active ingredient, at least about 1.0 wt. % of one or more surfactants, and water. The active ingredient comprises one or more of a probiotic, a probiotic derivative, and prebiotic. The topical composition reduces IL-8 concentration by a statistically significant amount, as compared to an otherwise identical composition without the active ingredient.

The topical composition decreases the concentration of IL-8 by at least about 30%, relative to an otherwise identical topical composition without the active ingredient.

Further exemplary embodiments relate to a topical cleansing composition for stimulating the production of antimicrobial peptides on the skin. The topical cleansing composition comprises about 0.005 wt. % to about 10.0 wt. % of an active ingredient, at least about 1.0 wt. % of one or more surfactants, and water. The active ingredient comprises one or more of a probiotic, a probiotic derivative, and prebiotic. The topical composition increases the concentration of antimicrobial peptides on skin by a statistically significant amount, as compared to an otherwise identical topical composition without the active ingredient.

In some exemplary embodiments, the topical cleansing composition increases the concentration of Involucrin by about 30%, relative to an otherwise identical topical composition without the active ingredient.

In some exemplary embodiments, the topical cleansing composition increases the concentration of HBD-2 by at least about 25%, relative to an otherwise identical topical composition without the active ingredient.

In some exemplary embodiments, the topical cleansing composition increases the concentration of DCS3 by at least about 25%, relative to an otherwise identical topical composition without the active ingredient.

Further exemplary embodiments relate to a topical lotion composition for restoring skin's natural balance of bacteria. The topical lotion composition comprises about 0.005 wt. % to 10.0 wt. % of an active ingredient comprising one or more of a probiotic, a probiotic derivative, and prebiotic, at least about 0.1 wt. % of an oil, about 0.01 wt. % to about 5.0 wt. % of a viscosity modifier, and water.

In some exemplary embodiments, the probiotic or probiotic derived ingredient is selected from a strain of one or more of Lactobacillus, Clostridia, Bifidobacterium, Saccharomyces, Lactococcus, Pedicoccus, Enterococcus, Escherichia, Alcaligenes, Corynebacterium, Bacillus, and Propionibacterium.

In some exemplary embodiments, the topical composition comprises about 0.2 to about 5 wt. %, or about 0.8 to about 1.5 wt. % active ingredient, based on the total weight of the topical composition.

### BRIEF DESCRIPTION OF THE FIGURES

FIG. 1 graphically illustrates the relative Interleukin 8 expression in topical compositions containing 1.0 wt. % Bonicel™ compared to a control.

FIG. 2 graphically illustrates the Involiucrin expression in compositions containing 1.0 wt. % Bonicel™ compared to a control.

FIG. 3 graphically illustrates the DSC3 expression in compositions containing 0.1 wt. % Bonicel™ compared to a control.

FIG. 4 graphically illustrates the HBD-2 expression in compositions containing 0.1 wt. % Bonicel™ and 1.0 wt. % Bonicel™ compared to a control.

FIG. 5 graphically illustrates the response of Staphylococcus aureus adhesion and invasion potential when treated with a probiotic Bacillus ferment.

### DETAILED DESCRIPTION

Unless otherwise defined, all technical and scientific terms used herein have the same meaning as commonly understood by one of ordinary skill in the art to which this application pertains. Although other methods and materials similar or equivalent to those described herein may be used in the practice or testing of the exemplary embodiments, exemplary suitable methods and materials are described below. In case of conflict, the present specification including definitions will control. In addition, the materials, methods, and examples are illustrative only and not intended to be limiting of the general inventive concepts.

The terminology as set forth herein is for description of the exemplary embodiments only and should not be construed as limiting the application as a whole. Unless otherwise specified, “a,” “an,” “the,” and “at least one” are used interchangeably. Furthermore, as used in the description of the application and the appended claims, the singular forms “a,” “an,” and “the” are inclusive of their plural forms, unless contradicted by the context surrounding such.

The term “microorganism” or “microbe” as used herein, refers to a tiny organism, such as a virus, protozoan, fungus, or bacterium that can only be seen under a microscope. The collection of microorganisms that live in an environment makes up a microbiota. For example human skin microbiota is all of the microbes on skin or a hospital microbiota would include all of the microbes in a hospital building. The term microbiome is used when referring to the entire habitat, including the microbiota as well as their genomes and the surrounding environment of the microbiota.

The phrase “topical composition” means a composition suitable for application directly to a surface, such as the surface of a human or animal body, including skin, and/or other surfaces, such as hair and nails.

The phrase “statistically significant” means p<0.05 for a test composition vs. a control that does not contain the active ingredient. The analysis is completed using 1) a T-test (a statistical examination of two population means) when only comparing one test article vs. one control); or 2) an analysis of variance (ANOVA) test when comparing two or more test articles vs. controls.

The general inventive concepts relate to a topical composition that contains an active ingredient that includes one or more of a probiotic or probiotic-derived ingredient and a prebiotic or prebiotic-derived ingredient. Generally, the active ingredient helps to restore skin's natural balance of bacteria. In some exemplary embodiments, the topical composition disclosed herein prevents pathogens from adhering to a surface, such as human skin or any inanimate surface. Such adherence prevention includes not only impeding the binding of a pathogen, but also promoting detachment of any already bound pathogen, and otherwise limiting the presence of such pathogens on a surface.

Some non-limiting examples of probiotic and probiotic-derived ingredients include strains and derivates of the following families: Actinomycetaceae, Corynebacteriaceae, Nocardiaceae, Intrasporangiaceae, Micrococcaceae, Propionibacteriacea, Bacteroidaceae, Porphyromonadaceae, Flavobacteriaceae, Sphingobacteriaceae, Bacillaceae, Exiguobacteraceae, Gemellaceae, Planococcaceae, Staphlococcaceae, Carnobacteriaceae, Aeorcoccaceae, Lactobacillaceae, Acidaminacoccaceae, Clostridiaceae, Lachnospiraceae, Peptostreptococcaceae, Veillonellaceae, Caulobactereaceae, Acetobacteraceae, Rhodobacteriaceae, Bradyrhizobiaceae, Brucellaceae, Sphingomonadaceae, Comamonadaceae, Neisseriaceae, Enterobaceriaceae, Pseudomonodaceae, Moraxellaceae, Pasteurellaceae, Xanthomonadaceae, Fusobacteriaceae, Chloroflexi, Chloroplasts, Cyanobacteria, and Streptophyta, for example. In some exemplary embodiments, the active ingredient is a probiotic or probiotic derived ingredient, which can be selected from a strain of one or more the following: Lactobacillus, strains and derivatives of Clostridia, strains and derivatives of Bifidobacterium, strains and derivatives of Saccharomyces, strains and derivatives of Lactococcus, strains and derivatives of Pedicoccus, strains and derivatives of Enterococcus, strains and derivatives of Escherichia, strains and derivatives of Alcaligenes, strains and derivatives of Corynebacterium, strains and derivatives of Bacillus, and strains and derivatives of Propionibacterium.

In some exemplary embodiments, the probiotic or probiotic derived ingredient is a ferment of Bacillus coagulans. Bacillus is a genus of Gram-positive, rod-shaped bacteria of the phylum Fimicutes. Bacillus can be either aerobic or, under certain conditions, anaerobic and produces endospores. Bacillus exhibits a wide range of physiologic properties that allows it to thrive in a number of different habitats—most Bacillus strains are resistant to heat, cold, radiation, and disinfectants. A Bacillus ferment (INCI name) is sold under the trade name Bonicel™ by Ganeden Biotech, Inc. in Cleveland, Ohio and is the supernatant produced by Bacillus coagulans GBI-30, 6086 (collectively referred to herein as “Bonicel™”). Bonicel™ is produced though a fermentation process which ensures the formulation includes the maximum amounts of enzymes, bateriocins, and L+ Lactic acid. Additional probiotic or probiotic derived ingredients may include Qi601 from Quorum Innovations, Repair Complex CLR™, EcoSkin® from Solabia Group, Leucidal® Liquid SF from Active Micro Technologies, ProSynergen™ from Lonza, ProBioBalance CLR™ from CLR, Yogurtene® Balance from Lonza, Biodynes™ from Lonza, Bifidobacterium Longum Lysate,

Some non-limiting examples of prebiotic ingredients include oligosaccharides, alpha and beta-glucan oligosaccharides, galactooligosaccharides, xylooligosaccharide, lactulose, inulin, ginseng, black current extract, sugar-beet extract, aloe extract, almond extract, tea extract, garlic extract, bark extract, chicory extract, corn extract, nerolidol extract, bisabolol extract, farnesol, xylitol, and pectin. Additional prebiotic ingredients may include EmulGold™ Fibre by Kerry Ingredients, Genu® Explorer Pectin by CP Kelco, Orafti® from Beneo, VitaFiber™ from BioNeutra, Konjac Glucomannan Hydrolysates, and Oat Beta Glucan from VegeTech.

In some embodiments, the active ingredient functions to simulate the production of anti-microbial peptides (AMPs) and thereby increase the overall concentration of AMPs on the surface of the skin. AMPs comprise a wide range of natural and synthetic peptides that are made of oligopeptides containing a varying number of amino acids. AMPs are essential components of host defense against infections present in all domains of life. AMPs are produced by all complex organisms and have diverse and intricate antimicrobial activities. As a whole, these peptides demonstrate a broad range of antiviral and antibacterial activities through an array of modes of action. AMPs have been found to kill Gram-negative and Gram-positive bacteria, certain viruses, parasites and fungi. Some research suggests that they can also enhance the internal immunity of complex organisms against a broad range of bacteria and viruses. In addition to the innate immune system present in all animals, vertebrates evolved an adaptive immune system based on specific recognition of antigens. Increasing evidence suggests that AMPs released in response to an invasion of microbial can activate adaptive immunity by attracting antigen-presenting dendritic cells to the invasion site.

In some embodiments, the active ingredient helps to restore the microbial balance of bacteria on the skin. A human's skin microbiota includes resident skin microorganisms that are continuously present on the skin. The resident skin microorganisms are usually non-pathogenic and either commensals (not harmful to their host) or mutualistic (offer a benefit). Resident skin microorganisms are adapted to survive on skin and they eat, reproduce, and excrete, which has an effect on the skin. However, certain transient skin microorganisms may attempt to colonize the skin, which could upset a healthy microbiome. Such transient skin microorganisms may include pathogens, such as pathogenic bacteria, yeasts, viruses, and molds. The particular make-up of a human's microbiome may be different than the make-up of another human's. A resident skin microorganism on one person may be a transient on another.

While the skin naturally works to regulate the microbiota on the surface, the active ingredients disclosed herein have been found to help in regulating and restoring the skin's natural balance.

The topical composition may comprise up to about 10.0 weight percent (wt. %) of the active ingredient, or up to about 8.0 wt. %, or up to about 5.0 wt. %, or up to about 3.0 wt. %, or up to about 2.0 wt. % of the active ingredient, based upon the total weight of the composition.

The topical composition may comprise at least about 0.001 wt. % active ingredient, or at least about 0.005 wt. %, or at least about 0.01 wt. %, or at least about 0.05 wt. %, or at least about 0.1 wt. %, or at least about 0.5 wt. %, or at least about 1.0 wt. % of the active ingredient, based upon the total weight of the topical composition.

In some exemplary embodiments, the topical composition comprises about 0.005 to about 10.0 wt. % of the active ingredient, or from about 0.5 to about 5.0 wt. % of the active ingredient, or from about 0.5 to about 2.0 wt. % of the active ingredient, based upon the total weight of the topical composition. In one exemplary embodiment, the topical composition comprises about 0.8 to about 1.5 wt. % of the active ingredient, based on the total weight of the topical composition.

In some exemplary embodiments, the topical composition is used for application to the skin and may be in the form of a skin cleanser, skin moisturizer, skin protectant, a wipe, a lotion, a salve, foam, and a gel. The topical composition may be applied to the skin before, during, or after skin cleaning.

In some exemplary embodiments, the topical composition includes water in an amount quantum sufficit (q.s.). In some exemplary embodiments, the topical composition comprises at least about 1.0 wt. % water, in another embodiment the topical composition comprises at least about 10.0 wt. % water, in another embodiment, the topical composition comprises at least about 20.0 wt. % water, in another embodiment, the topical composition comprises at least about 30.0 wt. % water, in another embodiment, the topical composition comprises at least about 40.0 wt. % water, in another embodiment, the topical composition comprises at least about 50.0 wt. % water, and in yet another embodiment, the topical composition comprises at least about 60.0 wt. % water, and in still yet another embodiment, the topical composition comprises at least about 70.0 wt. % water. In other embodiments, the topical composition comprises from about 20.0 wt. % to about 30.0 wt. % water. In yet other embodiments, the topical composition comprises from about 20.0 to about 24.0 wt. % water. More or less water may be required in certain instances, depending particularly on other ingredients and/or the amounts thereof employed in the topical composition.

In some exemplary embodiments, the topical composition includes one or more humectants. Non-limiting examples of humectants include propylene glycol, hexylene glycol, 1,4-dihydroxyhexane, 1,2,6-hexanetriol, sorbitol, butylene glycol, caprylyl glycol, propanediol s, such as methyl propane diol, dipropylene glycol, triethylene glycol, glycerin (glycerol), polyethylene glycols, ethoxydiglycol, polyethylene sorbitol, and combinations thereof. Other humectants include glycolic acid, glycolate salts, lactate salts, urea, Jojoba wax PEG-120 esters (commercially available from FloraTech), hydroxyethyl urea, alpha-hydroxy acids, such as lactic acid, sodium pyrrolidone carboxylic acid, hyaluronic acid, chitin, and the like. In one exemplary embodiment, the humecant is a mixture of caprylyl glycol, sodium L-pyroglutamate (Sodium PCA), and glycerin.

Examples of polyethylene glycol humectants include PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, and PEG-800.

The humectant may be included in the topical composition in an amount up to about 20.0 wt. %, or up to about 15.0 wt. %, or up to about 12.0 wt. %, or up to about 10.0 wt. %, or up to about 8.0 wt. % or up to about 5.0 wt. %, or up to about 3.0 wt. %. In some exemplary embodiments, the humectant is included in an amount from about 0.001 wt. %, or from about 0.01 wt. %, or from about 0.05 wt. %, or from about 0.1 wt. %, or from about 0.5 wt. %, or from about 0.7 wt. %, or from about 1.0 wt. %, or from about 1.5 wt. %, or from about 2.0 wt. %, based upon the total weight of the composition. In one exemplary embodiment, the humectant is included in an amount from about 0.4 to about 3.0 wt. %, or from about 1.5 to about 2.0 wt. %, based upon the total weight of the composition.

In one or more embodiments, the topical composition includes one or more skin-conditioners or emollients. Non-limiting examples of suitable skin conditioners and emollients include aloe, vitamin E, vitamin E acetate (tocopheryl acetate), Vitamin B₃ (niacinamide), C₆₋₁₀ alkane diols, sodium salt of pyroglutamic acid (sodium PCA), PEG-7 glyceryl cocoate, coco-glucoside and/or glyceryl oleate (Lamisoft® PO), and polyquaternium, such as polyquaternium 10 and 39.

The skin-conditioner or emollient can be included in the topical composition in an amount from about 0.001 to about 5.0 wt. %, in other embodiments, from about 0.005 to about 3.5 wt. %, or from about 0.01 to about 3.0 wt. %, or from about 0.05 to about 2.5 wt. %, or from about 0.1 to about 2.0 wt. %, or from about 0.5 to about 1.5 wt. %, based upon the total weight of the composition.

In some exemplary embodiments, the topical composition further includes a carrier component, such as a base cleaner.

The topical composition may further comprise one or more deposition enhancers. A suitable deposition enhancer works unidirectionally and will allow ingredients within the composition to penetrate deeper into the stratum corneum whilst preventing the loss of materials from the skin. Advantageously, the deposition enhancer provides a cosmetically acceptable skin feel to the formulation.

In one or more embodiments, the deposition enhancers include one or more of surfactants, bile salts and derivatives thereof, chelating agents, and sulphoxides.

Some examples of acceptable deposition enhancers include hydroxypropyl methylcellulose, dimethyl sulphoxides (DMSO), DMA, DMF, 1-dodecylazacycloheptan-2-one (azone), pyrrolidones such as 2-Pyrrolidone (2P) and N-Methyl-2-Pyrrolidone (NMP), long-chain fatty acids such as oleic acid and fatty acids with a saturated alkyl chain length of about C₁₀-C₁₂, essential oils, terpenes, terpenoids, oxazolidinones such as 4-decyloxazolidin-2-one, sodium lauryl sulfate (SLS), sodium laureate, polysorbates, sodium glyacolate, sodium deoxycholate, caprylic acid, EDTA, phospholipids, C₁₂₋₁₅ Alkyl Benzoate, pentylene glycol, ethoxydiglycol, polysorbate-polyethylenesorbitan-monolaurate, and lecithin.

In one or more exemplary embodiments, the deposition enhancer is a quaternary ammonium compound such as polyquaternium-6, -7, -10, -22, -37, -39, -74 or -101.

The deposition enhancer may be included in the topical composition in an amount from about 0.005 wt. % to about 10.0 wt. %, in other embodiments, from about 0.01 wt. % to about 5.0 wt. %, and in other embodiments, from about 0.05 wt. % to about 3.0 wt. %, based upon the total weight of the composition.

In one or more exemplary embodiments, the deposition enhancer comprises a hydroxy-terminated polyurethane compound chosen from polyolprepolymer-2, polyolprepolymer-14, and polyolprepolymer-15. Polyolprepolymer-2 is sometimes referred to as PPG-12/SMDI copolymer. The polyurethane compound may be present in the topical composition in an amount from about 0.005 wt. % to about 5.0 wt. %, in other embodiments, from about 0.01 wt. % to about 3.0 wt. %, and in other embodiments, from about 0.05 wt. % to about 1.0 wt. %, based upon the total weight of the composition.

The topical composition may further comprise one or more preservatives. A preservative is a natural or synthetic ingredient that can be added to personal care products to prevent spoilage, such as from microbial growth or undesirable chemical changes. Typical cosmetic preservatives are classified as natural antimicrobials, broad-spectrum preservatives, or stabilizers.

Many different types of preservatives are envisioned as being applicable in the current topical composition. Non-limiting examples of preservatives include one or more of isothiazolinones, such as methylchloroisothiazolinone and methylisothiazolinone; parabens including butylparaben, propylparaben, methylparaben and germaben II; phenoxyetyhanol and ethylhexylglycerin, organic acids such as potassium sorbate, sodium benzoate and levulinic acid; and phenoxyethanols.

The preservative can be added in the topical composition in an amount up to about 10.0 wt. %, preferably from about 0.05 wt. % to about 5.0 wt. %, more preferably from about 0.1 wt. % to about 2.0 wt. %, based on the weight of the total composition. In one exemplary embodiment, the preservative is present in an amount from about 1.0 to about 1.5 wt. %, based on the weight of the total composition.

The topical composition may further comprise one or more anti-irritants. Anti-irritants reduce signs of inflammation on the skin such as swelling, tenderness, pain, itching, or redness. There are three main types of anti-irritants, all of which are envisioned as being applicable in the subject invention: (1) compounds that operate by complexing the irritant itself, (2) compounds that react with the skin to block reactive sites preventing the irritant from reacting directly with the skin, and (3) compounds that prevent physical contact between the skin and irritant.

Some exemplary examples of suitable anti-irritants include Aloe Vera, allantoin, anion-cation complexes, aryloxypropionates, azulene, carboxymethyl cellulose, cetyl alcohol, diethyl phthalate, Emcol E607, ethanolamine, glycogen, lanolin, N-(2-Hydroxylthyl) Palmitamide, N-Lauroyl Sarcosinates, Maypon 4C, mineral oils, miranols, Myristyl lactate, polypropylene glycol, polyvinyl pyrrolidone (PVP), tertiary amine oxides, thiodioglycolic acid, and zirconia. In one exemplary embodiment, the anti-irritant is avenanthrmides (avena sativa (oat), kernel oil, and glycerin) and niacinamide.

The anti-irritant may be included in the topical composition in an amount up to about 10.0 wt. %, in other embodiments, from about 0.005 wt. % to about 3.0 wt. %, and in other embodiments, from about 0.01 wt. % to about 1.0 wt. %, based upon the total weight of the composition.

The topical composition may further comprise a fragrance. Any scent may be used in the topical composition including, but not limited to, any scent classification on a standard fragrance chart, such as floral, oriental, woody, and fresh. Exemplary scents include cinnamon, clove, lavender, peppermint, rosemary, thyme, thieves, lemon, citrus, coconut, apricot, plum, watermelon, ginger and combinations thereof.

The fragrance can be included in the topical composition in an amount from about 0.005 wt. % to about 5.0 wt. %, in other embodiments, from about 0.01 wt. % to about 3.0 wt. %, and in other embodiments, from about 0.05 wt. % to about 1.0 wt. %, based upon the total weight of the composition. The fragrance can be any made of any perfume, essential oil, aroma compounds, fixatives, terpenes, solvents, and the like. In some exemplary embodiments, the essential oils may include, for example, one or more of Limonene, Citrus Aurantium Dulcis (Orange) Peel Oil, Eucalyptus Globulus Leaf Oil, Citrus Grandis (Grapefruit) Peel Oil, Linalool, Litsea Cubeba Fruit Oil, Lavandula Hybrida Oil, Abies Sibirica Oil, Mentha Citrata Leaf Extract, Coriandrum Sativum (Coriander) Fruit Oil, Piper Nigrum (Pepper) Fruit Oil, and Canarium Luzonicum Gum Nonvolatiles.

The topical composition may further comprise a wide range of optional ingredients that do not deleteriously affect the composition's ability to stimulate AMP concentration on the surface or the composition's ability to regulate the balance of bacteria on the skin. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the present invention. Examples of these functional classes include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, keratolytics, topical active ingredients, and the like.

The inventive coating compositions exhibit a pH in the range of from about 2.5 to about 12.0, or a pH in the range of from about 3.5 to about 8.5, or in the range of from about 4.0 and about 8.0. When necessary, a pH adjusting agent or constituent may be used to provide and/or maintain the pH of a composition. Exemplary pH adjusting agents include, but are not limited to, organic acids, such as citric acid, lactic acid, formic acid, acetic acid, proponic acid, butyric acid, caproic acid, oxalic acid, maleic acid, benzoic acid, carbonic acid, and the like.

The form of the composition of the present invention is not particularly limited. In one or more embodiments, topical compositions of the present invention may be formulated as a lotion, a foamable composition, a rinse-off soap composition, a thickened gel composition, or may be applied to a wipe.

In one or more embodiments, the topical composition is formulated as a foamable composition. One or more foam agents may optionally be included in the foamable composition.

Any foaming agent conventionally known and used may be employed in the topical composition. In one or more embodiments, the foam agent comprises a non-ionic foam agent such as decyl glucoside or an amphoteric foam agent such as cocamidopropylbetaine. In one or more embodiments, the amount of nonionic or amphoteric foam agent is from about 0.5 to about 3.5 wt. %, in other embodiments from about 1.0 to about 3.0 wt. %, based upon the total weight of the topical composition. In one or more embodiments, the amount of decyl glucoside or cocamidopropylbetaine is from about 0.5 to about 3.5 wt. %, in other embodiments from about 1.0 to about 3.0 wt. %, based upon the total weight of the topical composition.

In some exemplary embodiments, the foaming agents include one or more of silicone glycol and fluorosurfactants. Silicone glycols may be generally characterized by containing one or more Si—O—Si linkages in the polymer backbone. Silicone glycols include organopolysiloxane dimethicone polyols, silicone carbinol fluids, silicone polyethers, alkylmethyl siloxanes, amodimethicones, trisiloxane ethoxylates, dimethiconols, quaternized silicone glycols, polysilicones, silicone crosspolymers, and silicone waxes.

Examples of silicone glycols include dimethicone PEG-7 undecylenate, PEG-10 dimethicone, PEG-8 dimethicone, PEG-12 dimethicone, perfluorononylethyl carboxydecal PEG 10, PEG-20/PPG-23 dimethicone, PEG-11 methyl ether dimethicone, bis-PEG/PPG-20/20 dimethicone, silicone quats, PEG-9 dimethicone, PPG-12 dimethicone, fluoro PEG-8 dimethicone, PEG-23/PPG-6 dimethicone, PEG-20/PPG-23 dimethicone, PEG 17 dimethicone, PEG-5/PPG-3 methicone, bis-PEG-18 methyl ether dimethyl silane, bis-PEG-20 dimethicone, PEG/PPG-20/15 dimethicone copolyol and sulfosuccinate blends, PEG-8 dimethicone\dimmer acid blends, PEG-8 dimethicone\fatty acid blends, PEG-8 dimethicone\cold pressed vegetable oil\polyquaternium blends, random block polymers and mixtures thereof.

The amount of silicone glycol foam agent is not particularly limited, so long as an effective amount to produce foaming is present. In certain embodiments, the effective amount to produce foaming may vary, depending upon the amount of other ingredients that are present. In one or more embodiments, the composition includes at least about 0.002 wt. % of silicone glycol foam agent, based upon the total weight of the composition. In another embodiment, the composition includes at least about 0.01 wt. % of silicone glycol foam agent, based upon the total weight of the composition. In yet another embodiment, the composition includes at least about 0.05 wt. % of silicone glycol foam agent, based upon the total weight of the composition.

In some exemplary embodiments, the foam agent is present in an amount of from about 0.002 to about 4.0 wt. %, or in an amount of from about 0.01 to about 2.0 wt. %, based upon the total weight of the composition. It is envisioned that higher amounts may also be effective to produce foam. All such weights as they pertain to listed ingredients are based on the active level, and therefore, do not include carriers or by-products that may be included in commercially available materials, unless otherwise specified.

In other embodiments, it may be desirable to use higher amounts of foam agent. For example, in certain embodiments where the foaming composition of the present invention includes a cleansing product that is applied to a surface and then rinsed off, higher amounts of foam agent may be employed. In these embodiments, the amount of foam agent is present in amounts up to about 35.0 wt. %, based upon the total weight of the composition.

The topical composition of the present invention may be formulated as an aerosol or non-aerosol foamable composition. In some exemplary embodiments the topical composition is dispensed from an unpressurized or low-pressure dispenser which mixes the composition with air.

In one or more embodiments, the viscosity of the non-aerosol foamable composition is less than about 100 mPas, in one embodiment less than about 50 mPas, and in another embodiment less than about 25 mPas.

In one or more embodiments, the compositions of the present invention may be formulated as a lotion. As is known in the art, lotions include oil-in-water emulsions as well as water-in-oil emulsions, oil-water-oil, and water-oil-water. A wide variety of ingredients may be present in either the oil or water phase of the emulsion. That is, the lotion formulation is not particularly limited.

Compositions of the present invention may be characterized by reference to viscosity and/or rheological properties. In one or more embodiments, the viscosity may be expressed as a standard, single-point type viscosity, as measured on a Brookfield Digital viscometer at a temperature of about 20° C., using spindle T-D, heliopath, at a speed of 10 rpm. In one or more embodiments, the compositions may have a viscosity of from about 2000 to about 120,000 cPs.

In one or more embodiments, compositions of the present invention may be characterized as lotions, having a viscosity of less than about 120,000 centipoise (cPs), in other embodiments, less than about 100,000, and in other embodiments, less than about 75,000 cPs. In one or more embodiments, the lotion compositions may have a viscosity of from about 3000 to about 50,000 cPs, in other embodiments, from about 4000 to about 30,000 cPs.

Exemplary lotion formulations include those containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties.

In one or more embodiments, compositions of the present invention may be characterized as serum, having a viscosity of from about 2000 to about 3000 cPs.

In one or more embodiments, compositions of the present invention may be characterized as creams, having a viscosity of from about 30,000 to about 100,000 cPs, in other embodiments from about 50,000 to about 80,000 cPs.

In one or more embodiments, compositions according to the present invention are pourable at room temperature, i.e. a temperature in the range of from about 20 to about 25° C. In one or more embodiments, the lotion formulations are viscous enough to hold a shape or not flow for a desired period of time. In other embodiments, compositions of the present invention are creams or ointments, and are not pourable and do not flow at room temperature and will not conform to a container when placed into the container at room temperature.

In one or more embodiments, the topical composition of the present invention may include thickeners and optionally one or more stabilizers. Examples of thickeners and stabilizers include polyurethane-based thickeners, such as steareth-100/PEG-136/HDI copolymer (Rheoluxe® 811); sodium chloride; propylene glycol; PEG-120 methyl glucose dioleate and methyl gluceth-10 (Ritathix DOE, available from Rita Corp.); hydroxyethyl cellulose; quaternized hydroxyethyl cellulose (Polyquaternium-10); hydroxypropyl cellulose; methyl cellulose; carboxymethyl cellulose; and ammonium acryloyldimethyltaurate/VP copolymer.

In one or more exemplary embodiments, the topical composition may be thickened with polyacrylate thickeners such as those conventionally available and/or known in the art. Examples of polyacrylate thickeners include carbomers, acrylates/C 10-30 alkyl acrylate cross-polymers, copolymers of acrylic acid and alkyl (C5-C 10) acrylate, copolymers of acrylic acid and maleic anhydride, and mixtures thereof. In one or more embodiments, the gel composition includes an effective amount of a polymeric thickener to adjust the viscosity of the gel to a viscosity range of from about 1000 to about 65,000 centipoise. In one embodiment, the viscosity of the gel is from about 5,000 to about 35,000, and in another embodiment, the viscosity is from about 10,000 to about 25,000. The viscosity is measured by a Brookfield RV Viscometer using RV and/or LV Spindles at 22° C.+/−3° C.

As will be appreciated by one of skill in the art, the effective amount of thickener will vary depending upon a number of factors, including the amount of other ingredients in the topical composition. In one or more embodiments, an effective amount of thickener is at least about 0.01 wt. %, based upon the total weight of the composition. In other embodiments, the effective amount is at least about 0.02 wt. %, or at least about 0.05 wt. %, or at least about 0.1 wt. %. In some exemplary embodiment, the effective amount of thickener is at least about 0.5 wt. %, or at least about 0.75 wt. %, based upon the total weight of the composition. In one or more embodiments, the compositions according to the present invention comprise up to about 10% by weight of the total composition of a polymeric thickener. In certain embodiments, the amount of thickener is from about 0.01 to about 1.0 wt. %, or from about 0.02 to about 0.4 wt. %, or from about 0.05 to about 0.3 wt. %, based upon the total weight of the composition. The amount of thickener may be from about 0.1 to about 10.0 wt. %, or from about 0.5 to about 5.0 wt. %, or from about 0.75 to about 2.0 wt. %, based upon the total weight of the composition.

In one or more embodiments, the topical composition may further comprise a neutralizing agent. Examples of neutralizing agents include amines, alkanolamines, alkanolamides, inorganic bases, amino acids, including salts, esters and acyl derivatives thereof. Exemplary neutralizing agents include triethanolamine, sodium hydroxide, monoethanolamine and dimethyl stearylamine. Other neutralizing agents are also known, such as HO(CₘH₂ₘ)₂NH, where m has the value of from 2 to 3, and aminomethyl propanol, aminomethyl propanediol, and ethoxylated amines, such as PEG-25 cocamine, polyoxyethylene (5) cocamine (PEG-5 cocamine), polyoxyethylene (25) cocamine (PEG-25 cocamine), polyoxyethylene (5) octadecylamine (PEG-5 stearamine), polyoxyethylene (25) octadecylamine (PEG-25 stearamine), polyoxyethylene (5) tallowamine (PEG-5 tallowamine), polyoxyethylene (15) oleylamine (PEG-15 oleylamine), polyethylene (5) soyamine (PEG-5 soyamine), and polyoxyethylene (25) soyamine (PEG-15 soyamine). A number of these are commercially available under the trade name of Ethomeen® from Akzo Chemie America, Armak Chemicals of Chicago, Ill.

In some exemplary embodiments the neutralizing agent includes at least one of sodium hydroxide or sodium hydroxide precursors. Solutions of sodium hydroxide in water are non-limiting examples of neutralizers containing sodium hydroxide.

The neutralizing agent is employed in an effective amount to neutralize a portion of the carboxyl groups of the thickening agent, and produce the desired pH range. The pH of un-neutralized thickening agent dispersed in water is generally acidic. For example, the pH of Carbopol® polymer dispersions is approximately in the range of 2.5 to 3.5, depending upon the polymer concentration. An effective amount of neutralizing agent, when added to the thickener dispersion, adjusts the pH to a desired range of about 4.1 to 4.8, or of about 4.2 to 4.6. The amount of neutralizing agent necessary to effect this pH range will vary depending upon factors such as the type of thickening agent, the amount of thickening agent, etc. However, in general, amounts less than 1.0 wt. % or ranging from about 0.001 to about 0.3 wt. % by weight of the neutralizing agent are considered sufficient and effective.

In some exemplary embodiments the topical composition can also be formulated as a soap. A fatty acid or a fatty acid ester may be used in conjunction with an alkali or base from the water phase to form a soap which has good water solubility as well as oil solubility properties and hence, is an excellent emulsifier. The soap, as explained above, can be in the form of a lotion soap, a foam soap, or any other common form known to one of skill in the art. Typical commercial blends such as oleic fatty acid, coconut fatty acid, soya fatty acid and tall oil fatty acid can be used. Preferably, the fatty acid comprises from about 5.0 to about 10.0 wt. % of the total topical composition.

As explained above, a base may be utilized in conjunction with the fatty acid to produce a soap on an equivalent basis of from about 2.7 to 0.8 equivalents to 1 equivalent of base. Examples of suitable base include organic alkalis or amines such as monoethanolamine, triethanolamine, and mixed isopropanolamines such as diisopropanolamine. Examples of suitable base also include inorganic alkalis, such as potassium hydroxide, sodium hydroxide, ammonium hydroxide, soda ash, and ammonia.

In addition, one or more non-fatty acid soap surfactants can be included in the oil phase of the cleaning composition in amounts preferably ranging up to about 25.0 wt. %. A surfactant is generally any substance which reduces the surface tension of a liquid. They break down the interface between water and oils/dirt. By holding the oils/dirt in suspension, they can be easily removed from the surface (i.e. skin).

In some exemplary embodiments, the surfactant includes a mixture of primary and secondary surfactants. Nonionic surfactants, i.e., surfactants which are uncharged (neutral) and without cationic or anionic sites, are preferred since they tend to render the composition stable, i.e., impart two desirable properties thereto. The first property is that of a suitable long shelf life. In other words, the emulsion can be held together at room temperature for long periods of time. The second desirable property is that upon use of the cleaning composition, the surfactant permits breakage of the emulsion or opening up thereof such that the hydrocarbon oil is readily released. The surfactant can also be an anionic surfactant, which carry a negative charge and are ionized in solution. The surfactant can also be a cationic surfactant, which carry a positive charge and ionize in solution. The surfactant can also be an amphoteric surfactant, which have the ability to be anionic (negatively charged), cationic (positively charged), or nonionic (uncharged, neutral) in solution depending on the pH.

It will be appreciated by one skilled in the art that in one or more embodiments, surfactant and/or surfactant combinations may be chosen to limit irritation of the composition and/or to enhance the effect of the active ingredient. In yet another embodiment, surfactant and/or surfactant combinations may be chosen to allow maximum bioavailability of the active ingredient. Non-limiting exemplary examples of surfactant combinations, levels of which will be known to one skilled in the art, are sodium lauryl ether sulfate (SLES) and/or cocamidopropyl betaine and/or disodium cocoamphodiacetate and/or surfactants of similar structure.

Non-limiting exemplary examples of surfactants that are envisioned in the present composition include betaines such as cocamidoproyl betaine; sulfonates and sulfates such as sodium laureth sulfate, sodium cocosulfate, sodium trideceth sulfate, and alkylbenzene sulfonate; glucosides, such as lauryl gluocoside and decyl glucoside; sodium cocoyl isothionate, sodium cocoyl glycinate, cocamidopropyl hydroxysultaine, PEG-80 sorbitan laurate, di-alkyl sulfosuccinate, lignosulfonates, disodium cocoamphodiacetate, lauryl glucoside, and PEG-80 sodium laurate.

In some exemplary embodiments, the topical cleansing composition comprises at least one primary surfactant and at least one secondary surfactant. A primary surfactant may include, for example, sodium laureth sulfate. Exemplary secondary surfactants may include, for example, one or more of cocamidopropyl betaine, disodium cocoamphodiacetate, cocamidopropyl hydroxysultaine, and lauryl glucoside.

The composition of the present invention may be employed in any type of dispenser typically used for gel products, for example pump dispensers. A wide variety of pump dispensers are suitable. Pump dispensers may be affixed to bottles or other free-standing containers. Pump dispensers may be incorporated into wall-mounted dispensers. Pump dispensers may be activated manually by hand or foot pump, or may be automatically activated. Useful dispensers include those available from GOJO Industries under the designations NXT® and TFX™ as well as traditional bag-in-box dispensers. Examples of dispensers are described in U.S. Pat. Nos. 5,265,772, 5,944,227, 6,877,642, 7,028,861, 7,611,030, and 7,621,426, all of which are incorporated herein by reference. In one or more embodiments, the dispenser includes an outlet such as a nozzle, through which the composition is dispensed. In some exemplary embodiments, the topical composition is used in dispensers that employ foaming pumps, which combine ambient air or an inert gas and the composition in a mixing chamber and pass the mixture through a mesh screen.

In one or more embodiments, the topical composition is integrated into wipe composition. Wipe compositions in accordance with this invention include at least one alcohol, a alkanediol enhancer, and are applied to a wipe substrate. In some exemplary embodiments, the wipe composition is alcohol-free.

Wipe substrates used in antimicrobial wipes are further described in U.S. Pat. Nos. 5,686,088, 6,410,499, 6,436,892, 6,495,508, 6,844,308. In one or more embodiments, the wipe may comprise a laminate formed by spunbonding/meltblowing/spunbonding (SMS). Generally, an SMS material contains a meltblown web sandwiched between two exteriors spunbond webs. SMS materials are further described in U.S. Pat. Nos. 4,041,203, 5,169,706, 5,464,688, and 4,766,029, and are commercially available, for example from Kimberly-Clark Corporation under marks such as Spunguard 7 and Evolution 7. The SMS laminate may be treated or untreated.

In some exemplary embodiments, the topical composition increases the production of Fillagrin, which is a biomarker known to correlate with improved skin health.

In some exemplary embodiments, the topical composition may reduce binding of MRSA in epithelial cells.

In some exemplary embodiments, the topical composition decreases the concentration of IL-8, a chmokine and proinflammatory cytokine. IL-8 is an important mediator of the immune reaction in the innate immune system response. Over-expressed IL-8 is a biomarker of skin irritation. IL-8 is associated with inflammation and plays a role in colorectal cancer. In some exemplary embodiments, a topical composition comprising up to about 10.0 wt. % of the active ingredient in water reduces the relative concentration of IL-8 by at least about 30%, or at least about 50%, or at least about 70%, or at least about 78% as compared to an otherwise identical control composition without the active ingredient.

In some exemplary embodiments, the topical composition increases the expression of Involucrin. Involucrin is a protein component of human skin and is encoded in humans by the IVL gene. In some exemplary embodiments, a topical composition comprising up to about 10.0 wt. % of an active ingredient is able to increase the relative Involucrin concentration by at least about 30% or at least about 50%, or at least about 70%, or at least about 90% or at least about 100% as compared to an otherwise identical composition not including the active ingredient.

In some exemplary embodiments, the topical composition increases the expression of DCS3. DSC3 is a calcium-dependent glycoprotein that is found in human epithelial cells and function as adhesives within the cell. In some exemplary embodiments, a topical composition comprising up to about 10.0 wt. % of an active ingredient is able to increase the relative DCS3 concentration by at least about 25%, or at least about 35%, or at least about 50%, or at least about 57%, as compared to an otherwise identical composition not including the active ingredient.

In some exemplary embodiments, topical composition comprising up to about 10.0 wt. % of an active ingredient increases the concentration of HBD-2. HBD-2 is a low molecular weight AMP produced by epithelia cells and is encoded by the DEFB4 gene. It exhibits potent antimicrobial activity against Gram-negative bacteria and Candida. HBD-2 plays an important role in the innate and adaptive immune system of both vertebrates and invertebrates. In humans it provides direct bactericidal action and Toll-like receptor activation.

In some exemplary embodiments, a topical composition comprising up to about 10.0 wt. % of an active ingredient in water is able to increase the relative concentration of HBD-2 by at least about 25%, or at least about 35%, or at least about 45%, or at least about 55%, or at least about 65%, or at least about 75%, or at least about 90%, or at least about 100%, or at least about 125%, or at least 140%, as compared to an otherwise identical control composition without the active ingredient.

### EXAMPLES

The following examples are included for purposes of illustration and are not intended to limit the scope of the methods described herein.

### Example 1

Topical compositions with Bonicel™ were tested for their ability to decrease concentration of Interleukin 8 (IL-8 or CXCL8) which is a chemokine and proinflammatory cytokine produced by macrophages and other cell types such as epithelial cells. IL-8 is secreted from keratinocytes in skin in response to inflammatory stimuli.

For Control A, human dermal keratinocytes were left untreated. No irritation is expected, and therefore Control A provides a baseline (set as 0). For Control B, IL-8 is induced in human dermal keratinocytes by applying a surfactant mixture that is a combination of sodium laureth sulfate and polyquaternium-10 (set as 100%). For all other samples, the human dermal keratinocytes are co-treated with the surfactant mixture and a composition containing indicated concentration of Bonicel™. Decreased IL-8 expression reflects an ingredient's anti-irritation activity. In order to carry out the test method, an assay kit was employed that was obtained from R&D Systems: Human CXCL8/IL-8 Duoset ELISA Kit (DY208). ELISA was performed after overnight treatment using by applying 100 μI/well of culture medium according to the manufactory instruction of the ELISA kit. The results were measured using a colorimeter, absorbance was measured at 450 nanometers (nm) within 30 minutes. Wavelength correction was set to 570 nm.

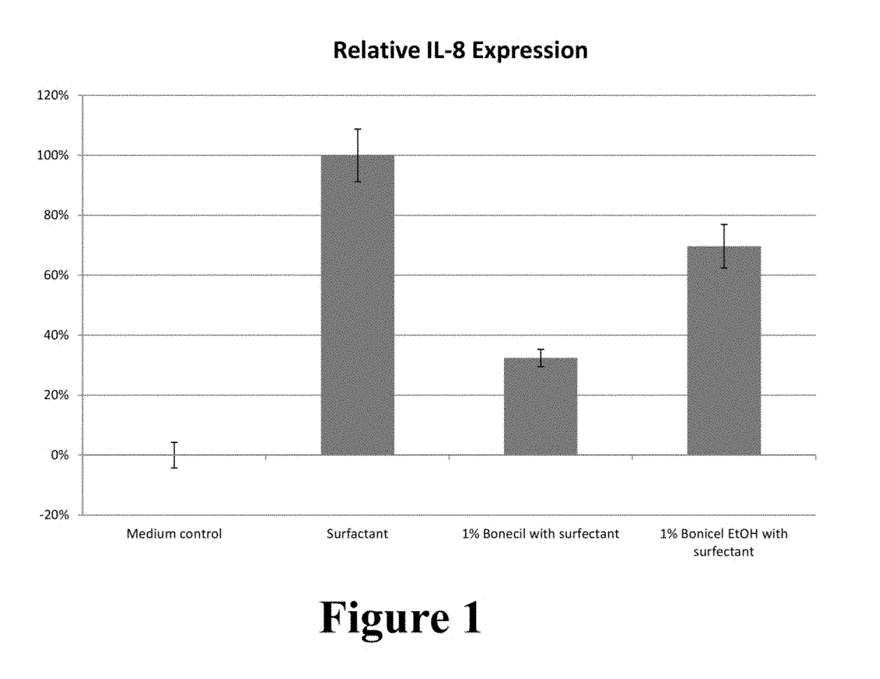

The results showed a topical composition with Bonicel™ was able to reduce the relative IL-8 expression. A relative decrease in IL-8 concentration of about 78% was observed for a topical composition with 1.0% Bonicel™, water, and a surfactant as compared to a control composition with water and a surfactant. The results are depicted graphically in FIG. 1.

### Example 2

An in vitro study was conducted to study a sample of Bonicel™ specifically for its ability to increase concentration of Involucrin.

Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). Keratinocytes were treated with the sample compositions in a 6-well plate overnight. After washing with cold phosphate-buffered saline (PBS), total RNAs were prepared from each well. Real-Time Quantitative Reverse Transcription PCR (qRT-PCR) was performed to detect the target genes (Involucrin) expression level using a One-step TaqMan® RT-PCR kit (Life Technologies).

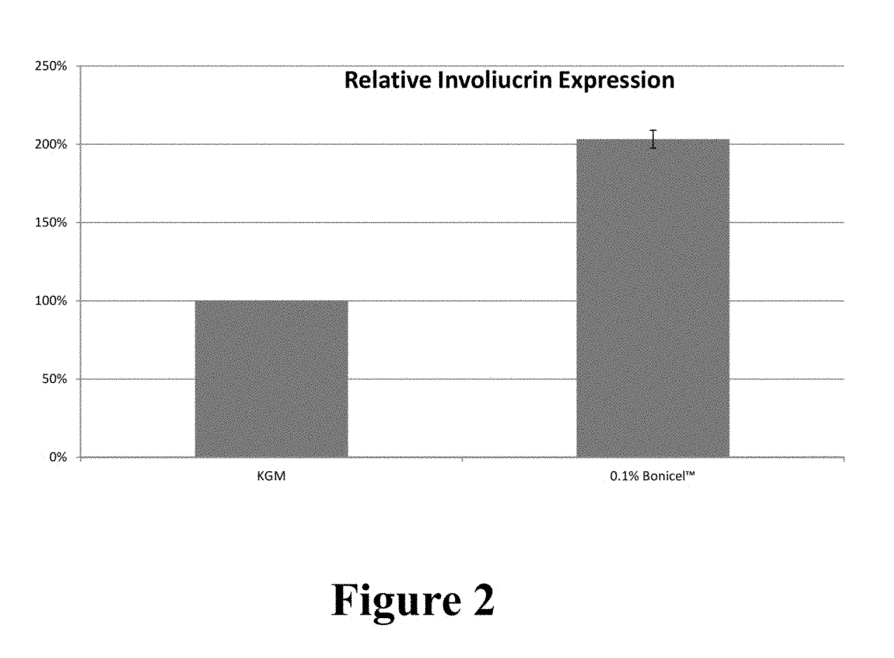

The results showed that Bonicel™ increased the relative expression of Involucrin. A relative increase in Involucrin concentration of 103% was observed for 0.1% Bonicel™ as compared to the KGM medium control culture. This increase shows that Bonicel™ can stimulate Involucrin in keratinocyte to promote skin keratinocyte differentiations and improve skin barrier function. The results are depicted graphically in FIG. 2.

### Example 3

An in vitro study was conducted to study a sample of Bonicel™ specifically for its ability to increase concentration of desmocollin-3 (DSC3). Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). Keratinocytes were treated with the sample compositions in a 6-well plate overnight. After washing with cold phosphate-buffered saline (PBS), total RNAs were prepared from each well. Real-Time Quantitative Reverse Transcription PCR (qRT-PCR) was performed to detect the target genes (DSC3) expression level using a One-step TaqMan® RT-PCR kit (Life Technologies).

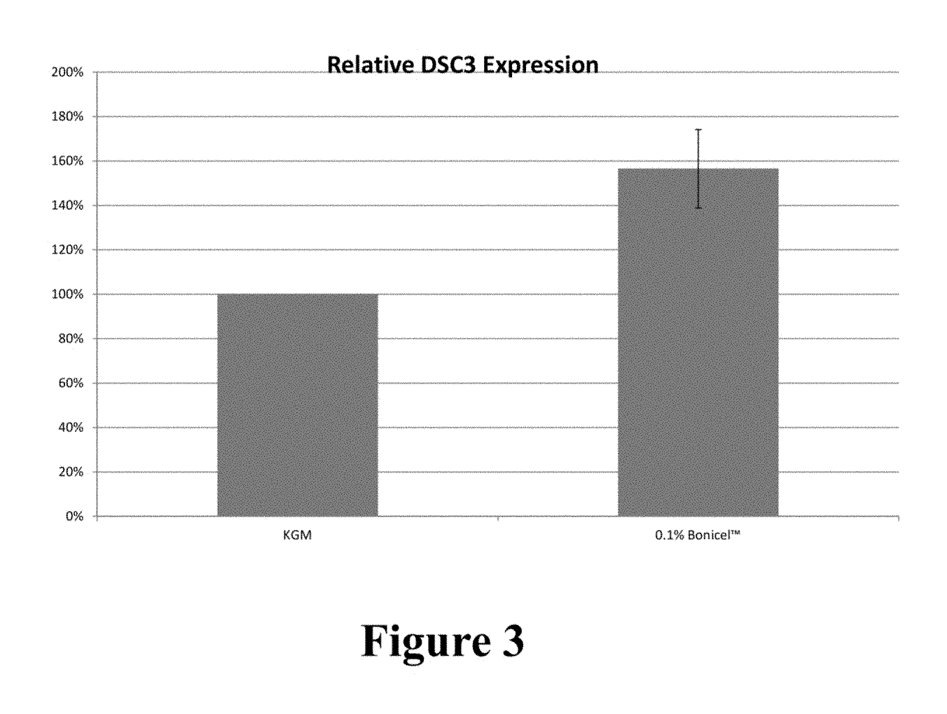

The results showed that Bonicel™ increased the relative expression of DSC3. A relative increase in DCS3 concentration of about 57% was observed for 0.1% Bonicel™ as compared to the KGM medium culture. This increase shows that Bonicel™ can stimulate skin junction biomarker DSC3 in keratinocytes to improve skin barrier function. The results are depicted graphically in FIG. 3.

### Example 4

In vitro studies were also run with Bonicel™ specifically to determine its ability to simulate growth in concentration of human beta-defensin 2 (HBD-2). Bonicel™ was tested at concentrations of both 0.1% and 1.0% in a water medium.

Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). NHEK were seeded into 96-well plates at a density of 10000 cells in 200 μl medium per well. After 48 hours, the cells were incubated with varying concentrations of each ingredient solution in a culture medium (KGM) overnight (16 hours) at 37° C., 5% CO2 and 95% humidity at four replicates for each concentration. Each of these active ingredients was tested at the different concentration of weight percents based on the weight of the total culture. Each of these compositions was compared to a control culture medium.

HBD-2 was detected using HBD-2 ELISA developing kits (commercially available from Peprotech). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO4 in each well. The results were measured using a colorimeter, absorbance was measured at 450 nanometers (nm) within 30 minutes. Wavelength correction was set to 570 nm. The concentration of each sample was calculated using ELISA standard curve.

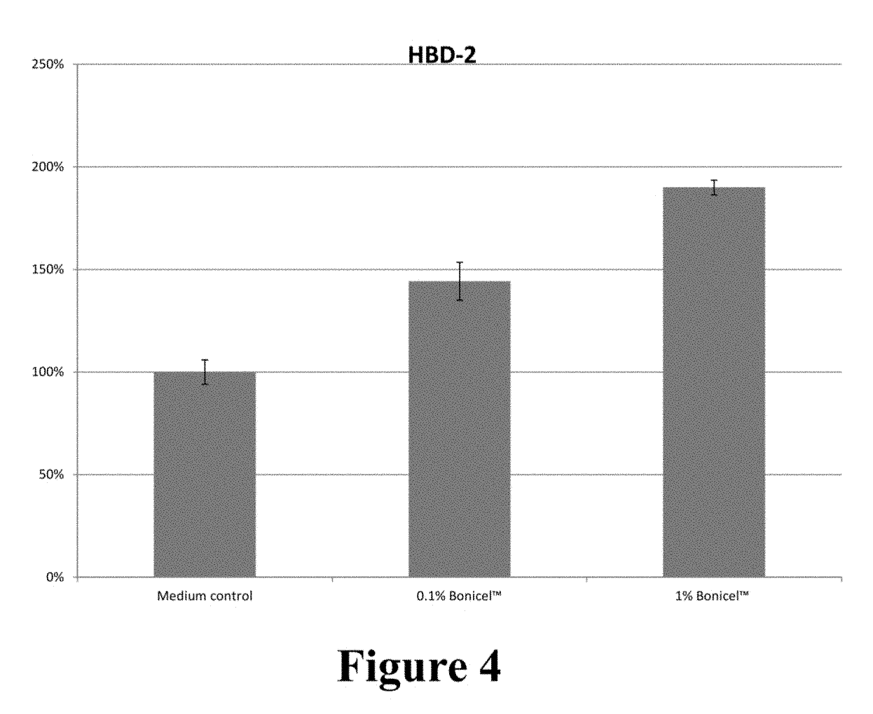

The results showed the Bonicel™ is able to increase the concentration of HBD-2 in a composition with water. Relative increases in HBD-2 concentration of about 44% and about 90% were observed for 0.1% Bonicel™ in a composition with water and 1.0% Bonicel™ in a composition with water, respectively. The results are depicted in FIG. 4.

### Example 5

The effect of exemplary topical compositions was investigated for pathogen blocking potential. Methicillin resistant Staphylococcus aureus strain Mu50 ATCC 33591, Escherichia coli strain K12 was tested against the following exemplary topical compounds: DMEM (cell culture medium, control), 100 nM dexamethasone (DEX, control steroidal anti-inflammatory), 0-5% Ecoskin (α-gluco-oligosaccharide, fructo-oligosaccharide and inactivated Lactobacillus), 0-5% Bacillus ferment, and 0-5% of a prebiotic blend of inulin and fructo-oligosaccahride.

Differentiated colonic epithelial cells were treated with the topical compounds and a bacterial strain was then added individually. The microbe was grown to the mid-log phase in an acceptable medium and the concentration adjusted so that the amount of bacteria added to the wells was approximately 100 microbes per well (in a 96 well tray with total volume of 100 uL). The cells were then incubated with each bacterial strain for one hour. A Gentamicin protection assay was used to determine adhered and invaded bacteria. Polymerase chain reaction (PCR) using 16S gene primers was used to determine the number of adhered bacteria, as well as the number of bacteria that invaded into the host cells.

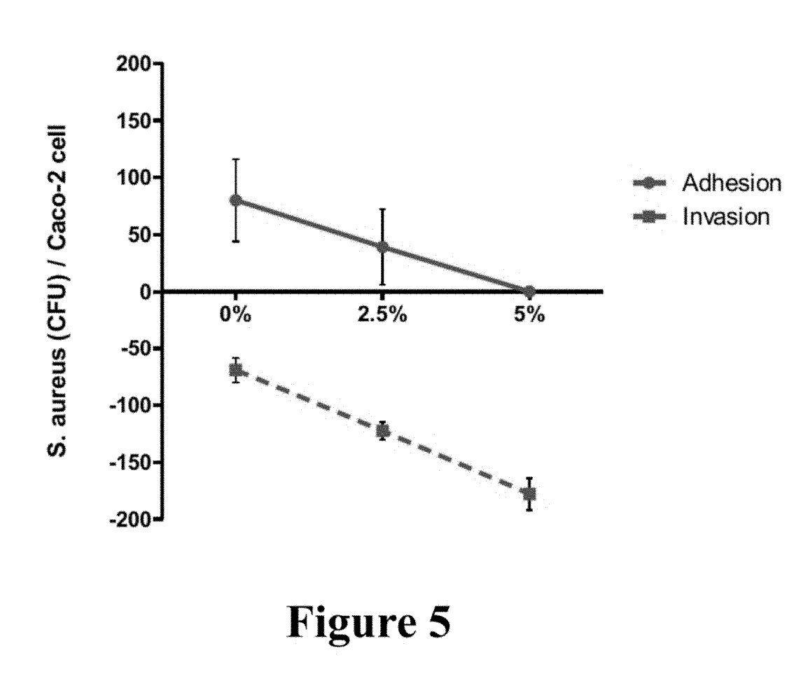

FIG. 5 illustrates the dose-dependent response of Staphylococcus aureus adhesion and invasion potential. Bacillus ferment had a consistent increase in the dose response. Particularly, 5% Bacillus ferment resulted in the lowest adhesion occurrence overall.

Although embodiments of the invention have been described herein, it should be appreciated that many modifications can be made without departing from the spirit and scope of the general inventive concepts. All such modifications are intended to be included within the scope of the invention.

1. A topical cleansing composition comprising:
about 0.005 wt. % to 10.0 wt. % of an active ingredient, comprising one or more of a probiotic, a probiotic derivative, and prebiotic;
about 0.1 wt. % to about 10.0 wt. % of a humectant;
at least about 1.0 wt. % of one or more surfactants; and
water, wherein the topical cleansing composition reduces pathogen binding on skin by a statistically significant amount, as compared to an otherwise identical topical composition without the active ingredient.

2. The topical cleansing composition of claim 2, wherein the active ingredient is a strain selected from a family selected from group consisting of: Actinomycetaceae, Corynebacteriaceae, Nocardiaceae, Intrasporangiaceae, Micrococcaceae, Propionibacteriacea, Bacteroidaceae, Porphyromonadaceae, Flavobacteriaceae, Sphingobacteriaceae, Bacillaceae, Exiguobacteraceae, Gemellaceae, Planococcaceae, Staphlococcaceae, Carnobacteriaceae, Aeorcoccaceae, Lactobacillaceae, Acidaminacoccaceae, Clostridiaceae, Lachnospiraceae, Peptostreptococcaceae, Veillonellaceae, Caulobactereaceae, Acetobacteraceae, Rhodobacteriaceae, Bradyrhizobiaceae, Brucellaceae, Sphingomonadaceae, Comamonadaceae, Neisseriaceae, Enterobaceriaceae, Pseudomonodaceae, Moraxellaceae, Pasteurellaceae, Xanthomonadaceae, Fusobacteriaceae, Chloroflexi, Chloroplasts, Cyanobacteria, and Streptophyta.

3. The topical cleansing composition of claim 1, wherein the probiotic or probiotic derived ingredient is selected from a strain of one or more of Lactobacillus, Clostridia, Bifidobacterium, Saccharomyces, Lactococcus, Pedicoccus, Enterococcus, Escherichia, Alcaligenes, Corynebacterium, Bacillus, and Propionibacterium.

4. The topical cleansing composition of claim 1, wherein the probiotic or probiotic derived ingredient is a ferment of Bacillus.

5. The topical cleansing composition of claim 4, wherein the ferment of Bacillus is Bonicel™.

6. The topical cleansing composition of claim 1, wherein the topical cleansing composition comprises 0.5 to 2 wt. % active ingredient.

7. The topical cleansing composition of claim 1, wherein the surfactant includes a mixture of primary and secondary surfactants.

8. The topical cleansing composition of claim 1, wherein said humectants is selected from the group consisting of propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, and combinations thereof.

9. A method of skin treatment for stimulating the production of antimicrobial peptides on the skin, said method comprising:
applying a topical cleansing composition to a skin surface, wherein the topical cleansing composition comprises:
about 0.005 wt. % to about 10.0 wt. % of an active ingredient, comprising one or more of a probiotic, a probiotic derivative, and prebiotic;
at least about 1.0 wt. % of one or more surfactants;
about 0.01 wt. % to about 10.0 wt. % of a humectant; and
water, wherein said topical cleansing composition reduces pathogen binding on skin by a statistically significant amount, as compared to an otherwise identical topical composition without the active ingredient.

10. The method of claim 9, wherein the method further includes rinsing the topical cleansing composition off with water.

11. The method of claim 9, wherein the probiotic or probiotic derived ingredient is a ferment of Bacillus.

12. A topical cleansing composition for reducing skin irritation, said composition comprising:
about 0.005 wt. % to about 10.0 wt. % of an active ingredient, comprising one or more of a probiotic, a probiotic derivative, and prebiotic;
at least about 1.0 wt. % of one or more surfactants; and
water, wherein the topical cleansing composition reduces IL-8 concentration by a statistically significant amount, as compared to an otherwise identical composition without the active ingredient.

13. The topical cleansing composition of claim 12, wherein the probiotic or probiotic derived ingredient is selected from a strain of one or more of Lactobacillus, Clostridia, Bifidobacterium, Saccharomyces, Lactococcus, Pedicoccus, Enterococcus, Escherichia, Alcaligenes, Corynebacterium, Bacillus, and Propionibacterium.

14. The topical cleansing composition of claim 12, wherein the probiotic or probiotic derived ingredient is a ferment of Bacillus.

15. A topical cleansing composition for stimulating the production of antimicrobial peptides on the skin, wherein the topical cleansing composition comprises:
about 0.005 wt. % to about 10.0 wt. % of an active ingredient, comprising one or more of a probiotic, a probiotic derivative, and prebiotic;
at least about 1.0 wt. % of one or more surfactants; and
water, wherein the topical cleansing composition increases the concentration of antimicrobial peptides on skin by a statistically significant amount, as compared to an otherwise identical topical composition without the active ingredient.

16. The topical cleansing composition of claim 15, wherein the topical cleansing composition increases the concentration of Involucrin by about 30%, relative to an otherwise identical topical composition without the active ingredient.

17. The topical cleansing composition of claim 15, wherein the topical cleansing composition increases the concentration of HBD-2 by at least about 25%, relative to an otherwise identical topical composition without the active ingredient.

18. The topical cleansing composition of claim 15, wherein the topical cleansing composition increases the concentration of DCS3 by at least about 25%, relative to an otherwise identical topical composition without the active ingredient.

19. A topical lotion composition for restoring skin's natural balance of bacteria, said topical lotion composition comprises:
about 0.005 wt. % to 10.0 wt. % of an active ingredient comprising one or more of a probiotic, a probiotic derivative, and prebiotic;
at least about 0.1 wt. % of an oil;
about 0.01 wt. % to about 5.0 wt. % of a viscosity modifier; and
water.

20. The topical lotion of claim 19, wherein the active ingredient is a strain selected from a group consisting of: Actinomycetaceae, Corynebacteriaceae, Nocardiaceae, Intrasporangiaceae, Micrococcaceae, Propionibacteriacea, Bacteroidaceae, Porphyromonadaceae, Flavobacteriaceae, Sphingobacteriaceae, Bacillaceae, Exiguobacteraceae, Gemellaceae, Planococcaceae, Staphlococcaceae, Carnobacteriaceae, Aeorcoccaceae, Lactobacillaceae, Acidaminacoccaceae, Clostridiaceae, Lachnospiraceae, Peptostreptococcaceae, Veillonellaceae, Caulobactereaceae, Acetobacteraceae, Rhodobacteriaceae, Bradyrhizobiaceae, Brucellaceae, Sphingomonadaceae, Comamonadaceae, Nei sseriaceae, Enterobaceriaceae, Pseudomonodaceae, Moraxellaceae, Pasteurellaceae, Xanthomonadaceae, Fusobacteriaceae, Chloroflexi, Chloroplasts, Cyanobacteria, and Streptophyta.

21. The topical lotion of claim 19, wherein the probiotic or probiotic derived ingredient is selected from a strain of one or more of Lactobacillus, Clostridia, Bifidobacterium, Saccharomyces, Lactococcus, Pedicoccus, Enterococcus, Escherichia, Alcaligenes, Corynebacterium, Bacillus, and Propionibacterium.

22. The topical lotion of claim 19, wherein the topical cleansing composition comprises 0.5 to 2 wt. % active ingredient.

23. The topical cleansing composition of claim 19, wherein the probiotic or probiotic derived ingredient is a ferment of Bacillus.
